(19) United States
(12) Patent Application Publication
(10) Pub. No.: US 2022/0125348 A1
(43) Pub. Date: Apr. 28, 2022

(54) DETECTION OF INTERFERENT IN ANALYTE SENSING

(71) Applicant: MEDTRONIC MINIMED, INC., Northridge, CA (US)

(72) Inventors: Steven C. Jacks, Culver City, CA (US); Ellis Garai, Studio City, CA (US)

(21) Appl. No.: 17/077,975

(22) Filed: Oct. 22, 2020

(51) Int. Cl. A61B 5/145; A61B 5/1468

(52) U.S. Cl. CPC A61B 5/14532; A61B 2560/0475; A61B 5/1468

(57) ABSTRACT

Methods for operating sensing devices, methods for correcting sensor glucose measurement signals, methods for detecting interferents in body fluid, and analyte monitoring apparatuses are provided. An exemplary method for operating a sensing device includes storing a library of changes in electrochemical impedance spectroscopy (EIS) signals correlated to known concentrations of the interferent within bodies of study subjects, wherein the library is accessible to a controller. The method also includes monitoring EIS signals of the user with the controller. Further, the method includes matching, with the controller, a change in an EIS signal of the user with a change in a selected EIS signal from the library. The method determines a concentration of an interferent within the body of a user based on the selected EIS signal from the library.

### TECHNICAL FIELD

Embodiments of the subject matter described herein relate generally to monitoring analyte levels in patients. More particularly, embodiments of the subject matter relate to operation of glucose sensors in conjunction with continuous glucose monitors and continuous glucose monitoring (CGM) to provide corrected glucose measurements despite the presence of an interferent.

### BACKGROUND

The pancreas of a normal healthy person produces and releases insulin into the blood stream in response to elevated blood plasma glucose levels. Beta cells (β-cells), which reside in the pancreas, produce and secrete insulin into the blood stream as it is needed. If β-cells become incapacitated or die, a condition known as Type 1 diabetes mellitus (or in some cases, if β-cells produce insufficient quantities of insulin, a condition known as Type 2 diabetes), then insulin may be provided to a body from another source to maintain life or health.

Traditionally, because insulin cannot be taken orally, insulin has been injected with a syringe. More recently, the use of infusion pump therapy has been increasing in a number of medical situations, including for delivering insulin to diabetic individuals. For example, external infusion pumps may be worn on a belt, in a pocket, or the like, and they can deliver insulin into a body via an infusion tube with a percutaneous needle or a cannula placed in subcutaneous tissue.

An infusion pump system may include an infusion pump that is automatically and/or semi-automatically controlled to infuse insulin into a patient. The infusion of insulin may be controlled to occur at times and in amounts that are based, for example, on blood glucose measurements obtained from an embedded analyte sensor, such as a glucose sensor, in real-time.

Analyte sensors such as biosensors include devices that use biological elements to convert a chemical analyte in a matrix into a detectable signal. There are many types of biosensors used for a wide variety of analytes. The most studied type of biosensor is the amperometric glucose sensor, which is crucial to the successful glucose level control for diabetes.

Errors in reading glucose levels may contribute to providing too much or too little insulin. For example, the presence of an endogenous or exogenous interferent in vivo at the monitoring site may cause gradual or incisive transgression toward an unsuitable glucose sensing environment. As a result, issues like current dips, sensor sensitivity loss, and false sensor glucose over-reading (increased current in response to the presence of an electroactive interferent) may occur.

### BRIEF SUMMARY

Methods for operating sensing devices, methods for correcting sensor glucose measurement signals, methods for detecting interferents in body fluid, and analyte monitoring apparatuses are provided. An exemplary method for operating a sensing device includes storing a library of changes in electrochemical impedance spectroscopy (EIS) signals correlated to known concentrations of the interferent within bodies of study subjects, wherein the library is accessible to a controller. The method also includes monitoring EIS signals of the user with the controller. Further, the method includes matching, with the controller, a change in an EIS signal of the user with a change in a selected EIS signal from the library. The method determines a concentration of an interferent within the body of a user based on the selected EIS signal from the library.

Methods for operating sensing devices, methods for correcting sensor glucose measurement signals, methods for detecting interferents in body fluid, and analyte monitoring apparatuses are provided. In an exemplary method for operating a sensing device associated with a user, the sensing device includes a controller coupled to a sensing element configured to measure a physiological condition in a body of the user. The exemplary method includes storing a library of changes in electrochemical impedance spectroscopy (EIS) signals correlated to known concentrations of an interferent within bodies of study subjects, wherein the library is accessible to the controller. Further, the method includes monitoring, by the controller, EIS signals of the user, and matching, by the controller, a change in an EIS signal of the user with a change in a selected EIS signal from the library. Also, the method includes determining a concentration of the interferent within the body of the user based on the selected EIS signal from the library.

In another exemplary method for operating a sensing device associated with a user, the sensing device includes a controller coupled to a sensing element configured to measure a physiological condition in a body of the user. The exemplary method includes transmitting a first voltage to the sensing device, monitoring a first sensor signal from the sensing device in response to the first voltage, and learning, with the controller, that an interferent is in a body fluid. The method includes, in response to learning that the interferent is in the body fluid, transmitting a second voltage to the sensing device, wherein the second voltage is less than the first voltage. Further, the method includes monitoring a second sensor signal from the sensing device in response to the second voltage.

In an exemplary embodiment, a method for correcting a sensor glucose measurement signal with a controller includes determining, with the controller, a concentration of an interferent in a body fluid. Further, the exemplary method includes modeling an effect on the sensor glucose measurement signal in response to the concentration of the interferent in the body fluid. Also, the exemplary method includes correcting the sensor glucose measurement signal based on the modeled effect.

An exemplary method for detecting the presence of an interferent in a body fluid includes contacting a sensing device with the body fluid, transmitting a voltage to the sensing device, and monitoring a sensor signal from the sensing device in response to the voltage. The exemplary method further includes calculating a sensor signal rate of change. The method includes identifying when the sensor signal rate of change is greater than a threshold indicative of the presence of the interferent in the body fluid.

In another embodiment, an analyte monitoring apparatus is provided. The exemplary analyte monitoring apparatus includes an electrochemical sensor for monitoring an electrochemical sensor placement site of a user, wherein the electrochemical sensor comprises a reference electrode; a counter electrode; and a working electrode. The apparatus further includes a sensor input configured to receive signals from the electrochemical sensor, and a processor coupled to the sensor input. The processor is configured to characterize one or more signals received from electrodes of the electrochemical sensor and to determine a concentration of acetaminophen at the electrochemical sensor placement site.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures.

FIG. 1 is a perspective view of a subcutaneous sensor insertion set and block diagram of a sensor electronics device according to an exemplary embodiment;

FIG. 2 illustrates a substrate having two sides, a first side which contains an electrode configuration and a second side which contains electronic circuitry;

FIG. 3 illustrates a general block diagram of an electronic circuit for sensing an output of a sensor;

FIG. 4 illustrates a block diagram of a sensor electronics device and a sensor including a plurality of electrodes according to an exemplary embodiment;

FIG. 5 illustrates an exemplary embodiment including a sensor and a sensor electronics device;

FIG. 6 illustrates an electronic block diagram of the sensor electrodes and a voltage being applied to the sensor electrodes according to an embodiment;

FIG. 7 illustrates an effect of an interferent, such as acetaminophen, on a glucose measurement for an exemplary glucose sensor according to an embodiment;

FIG. 8 illustrates an in vivo study of the concentration of acetaminophen in plasma and in interstitial fluid over time;

FIG. 9 illustrates the change in the imaginary component of the impedance (Zimag) in vivo over time after ingestion of acetaminophen;

FIG. 10 illustrates changes in imaginary component of the impedance in response to the presence of a known input of acetaminophen in an in vitro study;

FIG. 11 illustrates a system for correcting sensor glucose measurements based on a modeled response to the presence of an interferent according to an embodiment;

FIG. 12 illustrates an exemplary modeling process according to an embodiment;

FIG. 13 illustrates the percent change in the imaginary component of impedance (i.e., a normalized ΔZimag) in response to the presence of an interferent;

FIG. 14 illustrates an exemplary process for operating a sensing device according to an embodiment;

FIG. 15 illustrates an exemplary process for determining a concentration of interferent in the body according to an embodiment;

FIG. 16 illustrates an exemplary detection method and correction method according to an embodiment;

FIG. 17 illustrates an exemplary detection method and correction method according to another embodiment; and

FIGS. 18-20 illustrate the effect of an interferent on a sensor glucose signal as compared to accurate blood glucose measurements, and the correction of the sensor glucose signal according to an embodiment.

### DETAILED DESCRIPTION

The following detailed description is merely illustrative in nature and is not intended to limit the embodiments of the subject matter or the application and uses of such embodiments. It is understood that other embodiments may be utilized and structural and operational changes may be made without departing from the scope of the subject matter. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description. Also, while the preceding background discusses glucose sensing and exemplary analyte sensors are described as glucose sensors herein, such description is for convenience and is not limiting. The claimed subject matter may include any type of analyte sensor or method for measuring an analyte as described herein. Further, a glucose sensor may measure blood glucose directly from a blood stream, indirectly via interstitial fluid using for example a subcutaneous sensor, or some combination thereof. As used herein, “blood glucose”, “measured blood glucose”, “blood glucose concentration”, “measured blood glucose concentration”, and the like may refer to a glucose level, a blood glucose level, a blood glucose concentration, and so forth that has been obtained via any type of glucose sensor. It should be understood, however that using a blood glucose sensor is only one particular technique for obtaining such observations or measurements, and that other techniques, such as measuring blood glucose from observations of other body fluids (e.g., observations of the presence of glucose in interstitial fluid using a subcutaneous sensor), may be used without deviating from claimed subject matter.

In an exemplary analyte monitoring apparatus, blood-glucose measurements may be employed in a closed loop infusion system for regulating a rate of fluid infusion into a body. In particular embodiments, a control system may be adapted to regulate a rate of insulin, glucagon, and/or glucose infusion into a body of a patient based, at least in part, on a glucose concentration measurement taken from a body (e.g., from a glucose sensor).

According to exemplary embodiments, examples of analyte sensors and/or monitoring apparatuses as described herein may be implemented in a hospital environment to monitor levels of glucose in a patient. Alternatively, according to certain embodiments, examples of analyte sensors and/or monitoring apparatuses as described herein may be implemented in non-hospital environments to monitor levels of glucose in a patient. Here, a patient or other non-medical professional may be responsible for interacting with analyte sensors and/or monitoring apparatuses.

To maintain healthy glucose levels, a person with type 1 diabetes may manage their glycemia by monitoring blood glucose levels, controlling diet, exercise, and self-administering appropriate amounts of insulin at appropriate times. Deviations from such glycemic management, such as skipping an insulin bolus at mealtime or underestimating the carbohydrate content of a meal may bring about prolonged hyperglycemia. Likewise, receiving too much insulin (e.g., by over-bolusing) for a given blood glucose level and/or meal may bring about severe hypoglycemia. Other external factors, such as exercise or stress, may also contribute to glycemic deviations.

Further, errors in reading glucose levels may be caused by endogenous or exogenous interferents in vivo at the monitoring site. Various solutions have been proposed to circumvent errors caused by exogenous interferents directly reacting with the surface of glucose sensor electrodes. For example, in some instances, devices have utilized an additional sensor or an additional working electrode to identify and subtract noise from the primary signal to generate the Sensor Glucose (SG) value. In other cases, an interferent rejection membrane, such as a size exclusion membrane, is located around the working electrode to block the interferent from reacting with the working electrode.

While these methods may work to overcome the effects of interferents in the interstitial fluid, they require the addition of materials and complexity to the sensor systems. There is a need for improved methods and systems for monitoring analyte levels in patients that reduce measurement errors induced by interferents without requiring any additional materials or devices.

An exemplary embodiment of an analyte sensor or monitoring apparatus has the diagnostic capability to detect or characterize transient incisive but false increase in sensor glucose value due to introduction of electroactive interferents. Such embodiments may reduce the risk of hypoglycemia and hyperglycemia by eliminating or reducing analyte monitoring error. In the context of this description, an “interferent” is a substance that interferes with the ability of the analyte sensor to measure the analyte. Electroactive interferents may include, without limitation, acetaminophen, dopamine, gentisic acid, ibuprofen, L-DOPA, methyldopa, and the like. In some examples, one or more of these interferents may be administered to the patient by medical personnel at a hospital or clinic.

By more accurately monitoring a patient's glucose level and maintaining appropriate infusion rates, extreme glycemic variations may be reduced or avoided altogether. This may provide a patient with improved glycemic control in circumstances in which they would otherwise be exposed to undesirable extremes of glycemia.

In exemplary embodiments, the subject matter herein provides for the detection of an interferent, such as acetaminophen, in a body fluid at a sensor placement location. The interferent presence and/or concentration may be determined by using signals from the working electrode of the analyte sensor, for example, Electrochemical Impedance Spectroscopy (EIS) signals tuned specifically to the low frequency region of the EIS. In certain embodiments, the interferent presence and/or concentration may be determined by calculating a sensor signal rate of change, such as a Sensor Glucose (SG) rate of change. Further, exemplary embodiments may take EIS signals, and/or sensor signal rate of change, as well as changes in other signals such as counter voltage signals (Vctr) and current signals (Isig), to determine interferent presence and/or concentration.

Also, embodiments herein provide for using the changes in these selected signals within a model to correct for the sensor glucose error that would otherwise be observed due to the interferent. In other words, embodiments herein include building a model to correct for any changes that would be observed and otherwise lead to an inherent bias in the final sensor glucose output. As a result, the bias due to the interferent is mitigated.

Other embodiments provide for the modulation of a voltage transmitted to the sensing device in response to the detection of an interferent. For example, after detecting the interferent the voltage transmitted to the sensing device may be changed. Specifically, the voltage may be reduced to a lower voltage. In certain embodiments, the voltage may be pulse modulated such that a first voltage and a second voltage are applied successively to the sensing device for selected durations of time until the interferent is no longer detected or is detected at a sufficiently low level.

The subject matter herein may be described with reference to flowchart illustrations of methods, systems, devices, apparatus, and programming and computer program products. It will be understood that each block of the flowchart illustrations, and combinations of blocks in the flowchart illustrations, can be implemented by programming instructions, including computer program instructions (as can any menu screens described in the figures). These computer program instructions may be loaded onto a computer or other programmable data processing apparatus (such as a controller, microcontroller, or processor in a sensor electronics device) to produce a machine, such that the instructions which execute on the computer or other programmable data processing apparatus create instructions for implementing the functions specified in the flowchart block or blocks. These computer program instructions may also be stored in a computer-readable memory that can direct a computer or other programmable data processing apparatus to function in a particular manner, such that the instructions stored in the computer-readable memory produce an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks, and/or menus presented herein. Programming instructions may also be stored in and/or implemented via electronic circuitry, including integrated circuits (ICs) and Application Specific Integrated Circuits (ASICs) used in conjunction with sensor devices, apparatuses, and systems.

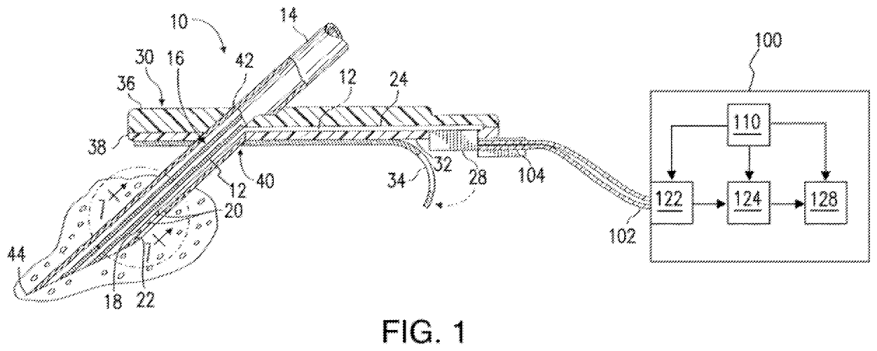

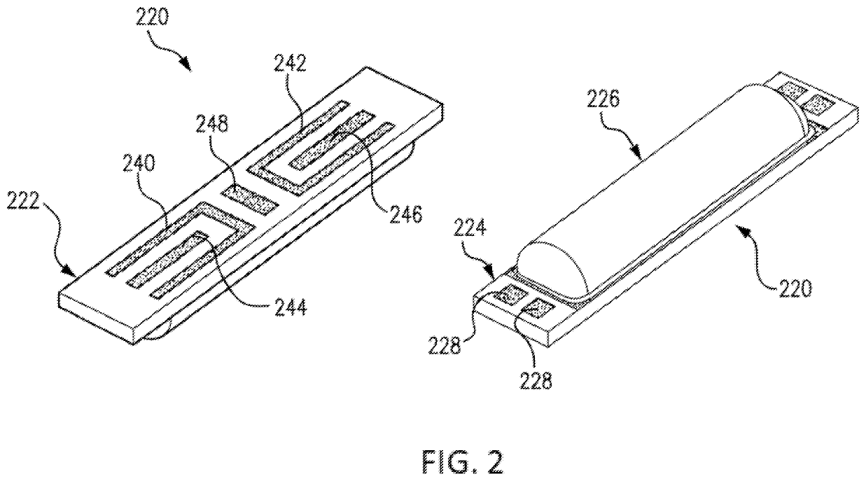

FIG. 1 is a perspective view of a subcutaneous sensor insertion set and a block diagram of a sensor electronics device according to an exemplary embodiment. As illustrated in FIG. 1, a subcutaneous sensor set 10 is provided for subcutaneous placement of an active portion of a flexible sensor 12 (see, e.g., FIG. 2), or the like, at a selected site in the body of a user. The subcutaneous or percutaneous portion of the sensor set 10 includes a hollow, slotted insertion needle 14, and a cannula 16. The needle 14 is used to facilitate quick and easy subcutaneous placement of the cannula 16 at the subcutaneous insertion site. Inside the cannula 16 is a sensing portion 18 of the sensor 12 to expose one or more sensor electrodes 20 to the user's bodily fluids, such as interstitial fluid or blood, through a window 22 formed in the cannula 16. In an embodiment, the one or more sensor electrodes 20 may include a counter electrode, a reference electrode, and one or more working electrodes. After insertion, the insertion needle 14 is withdrawn to leave the cannula 16 with the sensing portion 18 and the sensor electrodes 20 in place at the selected insertion site.

In particular embodiments, the subcutaneous sensor set 10 facilitates accurate placement of a flexible thin film electrochemical sensor 12 of the type used for monitoring specific blood parameters representative of a user's condition. The sensor 12 monitors glucose levels in the body, and may be used in conjunction with automated or semi-automated medication infusion pumps of the external or implantable type as described, e.g., in U.S. Pat. Nos. 4,562,751; 4,678,408; 4,685,903 or 4,573,994, to control delivery of insulin to a diabetic patient, which are herein incorporated by reference.

Exemplary embodiments of the flexible electrochemical sensor 12 are constructed in accordance with thin film mask techniques to include elongated thin film conductors embedded or encased between layers of a selected insulative material such as polyimide film or sheet, and membranes. The sensor electrodes 20 at a tip end of the sensing portion 18 are exposed through one of the insulative layers for direct contact with patient blood or other body fluids, when the sensing portion 18 (or active portion) of the sensor 12 is subcutaneously placed at an insertion site. The sensing portion 18 is joined to a connection portion 24 that terminates in conductive contact pads, or the like, which are also exposed through one of the insulative layers. In exemplary embodiments, other types of implantable sensors, such as chemical based, optical based, or the like, may be used.

As is known in the art, the connection portion 24 and the contact pads are generally adapted for a direct wired electrical connection to a suitable monitor or sensor electronics device 100 for monitoring a user's condition in response to signals derived from the sensor electrodes 20. Further description of flexible thin film sensors of this general type can be found in U.S. Pat. No. 5,391,250, which is herein incorporated by reference. The connection portion 24 may be conveniently connected electrically to the monitor or sensor electronics device 100 or by a connector block 28 (or the like) as shown and described in U.S. Pat. No. 5,482,473, which is also herein incorporated by reference. Thus, in accordance with embodiments, subcutaneous sensor sets 10 may be configured or formed to work with either a wired or a wireless characteristic monitor system.

The sensor electrodes 20 may be used in a variety of sensing applications and may be configured in a variety of ways. For example, the sensor electrodes 20 may be used in physiological parameter sensing applications in which some type of biomolecule is used as a catalytic agent. For example, the sensor electrodes 20 may be used in a glucose and oxygen sensor having a glucose oxidase (GOx) enzyme catalyzing a reaction with the sensor electrodes 20. The sensor electrodes 20, along with a biomolecule or some other catalytic agent, may be placed in a human body in a vascular or non-vascular environment. For example, the sensor electrodes 20 and biomolecule may be placed in a vein and be subjected to a blood stream, or may be placed in a subcutaneous or peritoneal region of the human body.

The monitor 100 may also be referred to as a sensor electronics device 100. The monitor 100 may include a power source 110, a sensor interface 122, processing electronics 124, and data formatting electronics 128. The processing electronics 124 and data formatting electronics 128 may be considered to form a controller. The monitor 100 may be coupled to the sensor set 10 by a cable 102 through a connector that is electrically coupled to the connector block 28 of the connection portion 24. In an exemplary embodiment, the cable may be omitted. In this embodiment, the monitor 100 may include an appropriate connector for direct connection to the connection portion 104 of the sensor set 10. The sensor set 10 may be modified to have the connector portion 104 positioned at a different location, e.g., on top of the sensor set to facilitate placement of the monitor 100 over the sensor set.

In exemplary embodiments, the sensor interface 122, the processing electronics 124, and the data formatting electronics 128 are formed as separate semiconductor chips, however, exemplary embodiments may combine the various semiconductor chips into a single or multiple customized semiconductor chips. The sensor interface 122 connects with the cable 102 that is connected with the sensor set 10.

The power source 110 may be a battery. The battery can include three series silver oxide 357 battery cells. In exemplary embodiments, different battery chemistries may be utilized, such as lithium based chemistries, alkaline batteries, nickel metalhydride, or the like, and a different number of batteries may be used. The monitor 100 provides power to the sensor set via the power source 110, through the cable 102 and cable connector 104. In an exemplary embodiment, the power is a voltage provided to the sensor set 10. In an exemplary embodiment, the power is a current provided to the sensor set 10. In an exemplary embodiment, the power is a voltage provided at a specific voltage to the sensor set 10.

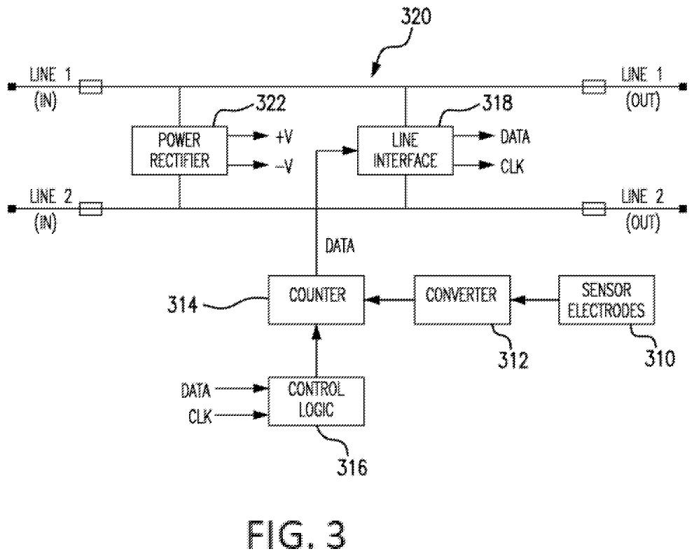

FIGS. 2 and 3 illustrate an implantable sensor and electronics for driving the implantable sensor according to an embodiment. FIG. 2 shows a substrate 220 having two sides, a first side 222 that contains an electrode configuration and a second side 224 that contains electronic circuitry. As may be seen in FIG. 2, a first side 222 of the substrate comprises two counter electrode-working electrode pairs 240, 242, 244, 246 on opposite sides of a reference electrode 248. A second side 224 of the substrate comprises electronic circuitry. As shown, the electronic circuitry may be enclosed in a hermetically sealed casing 226, providing a protective housing for the electronic circuitry. This allows the sensor substrate 220 to be inserted into a vascular environment or other environment which may subject the electronic circuitry to fluids. By sealing the electronic circuitry in a hermetically sealed casing 226, the electronic circuitry may operate without risk of short circuiting by the surrounding fluids. Also shown in FIG. 2 are pads 228 to which the input and output lines of the electronic circuitry may be connected. The electronic circuitry itself may be fabricated in a variety of ways. According to an embodiment, the electronic circuitry may be fabricated as an integrated circuit using techniques common in the industry.

FIG. 3 illustrates a general block diagram of an electronic circuit for sensing an output of a sensor according to an embodiment. At least one pair of sensor electrodes 310 may interface to a data converter 312, the output of which may interface to a counter 314. The counter 314 may be controlled by control logic 316 (e.g., a controller). The output of the counter 314 may connect to a line interface 318. The line interface 318 may be connected to input and output lines 320 and may also connect to the control logic 316. The input and output lines 320 may also be connected to a power rectifier 322.

The sensor electrodes 310 may be used in a variety of sensing applications and may be configured in a variety of ways. For example, the sensor electrodes 310 may be used in physiological parameter sensing applications in which some type of biomolecule is used as a catalytic agent. For example, the sensor electrodes 310 may be used in a glucose and oxygen sensor having a glucose oxidase (GOx) enzyme catalyzing a reaction with the sensor electrodes 310. The sensor electrodes 310, along with a biomolecule or some other catalytic agent, may be placed in a human body in a vascular or non-vascular environment. For example, the sensor electrodes 310 and biomolecule may be placed in a vein and be subjected to a blood stream.

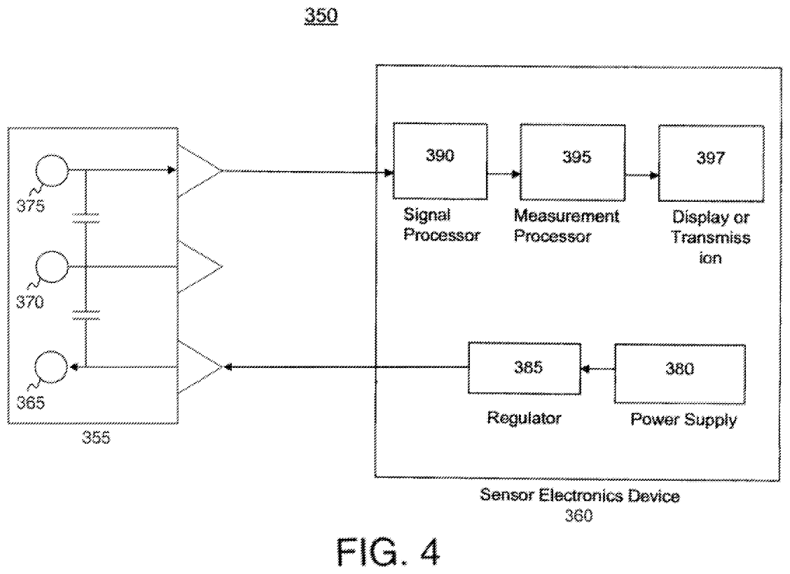

FIG. 4 illustrates a block diagram of a sensor electronics device and a sensor including a plurality of electrodes according to an embodiment. The sensor set or system 350 includes a sensor 355 and a sensor electronics device 360. The sensor 355 includes a counter electrode 365, a reference electrode 370, and a working electrode 375. The sensor electronics device 360 includes a power supply 380, a regulator 385, a signal processor 390, a measurement processor 395, and a display/transmitter 397. The power supply 380 provides power (in the form of either a voltage, a current, or a voltage including a current) to the regulator 385. The regulator 385 transmits a regulated voltage to the sensor 355. In an embodiment, the regulator 385 transmits a voltage to the counter electrode 365 of the sensor 355.

The sensor 355 creates a sensor signal indicative of a concentration of a physiological characteristic being measured. For example, the sensor signal may be indicative of a blood glucose reading. In an embodiment utilizing subcutaneous sensors, the sensor signal may represent a level of hydrogen peroxide in a subject. In an embodiment where blood or cranial sensors are utilized, the amount of oxygen is being measured by the sensor and is represented by the sensor signal. In an embodiment utilizing implantable or long-term sensors, the sensor signal may represent a level of oxygen in the subject. The sensor signal is measured at the working electrode 375. In an embodiment, the sensor signal may be a current measured at the working electrode. In an embodiment, the sensor signal may be a voltage measured at the working electrode.

The signal processor 390 receives the sensor signal (e.g., a measured current or voltage) after the sensor signal is measured at the sensor 355 (e.g., the working electrode). The signal processor 390 processes the sensor signal and generates a processed sensor signal. The measurement processor 395 receives the processed sensor signal and calibrates the processed sensor signal utilizing reference values. In an embodiment, the reference values are stored in a reference memory and provided to the measurement processor 395. The measurement processor 395 generates sensor measurements. The sensor measurements may be stored in a measurement memory (not shown). The sensor measurements may be sent to a display/transmission device to be either displayed on a display in a housing with the sensor electronics or transmitted to an external device.

The sensor electronics device 360 may be a monitor which includes a display to display physiological characteristics readings. The sensor electronics device 360 may also be installed in a desktop computer, a television including communications capabilities, a laptop computer, a server, a network computer, a personal digital assistant (PDA), a portable telephone including computer functions, an infusion pump including a display, a glucose sensor including a display, and/or a combination infusion pump/glucose sensor. The sensor electronics device 360 may be housed in a hand-held device (i.e., smartphone), wearable device (i.e., smartwatch), a network device, cloud, a home network device, a dedicated standalone disposable device, single-use package, or an appliance connected to a home network.

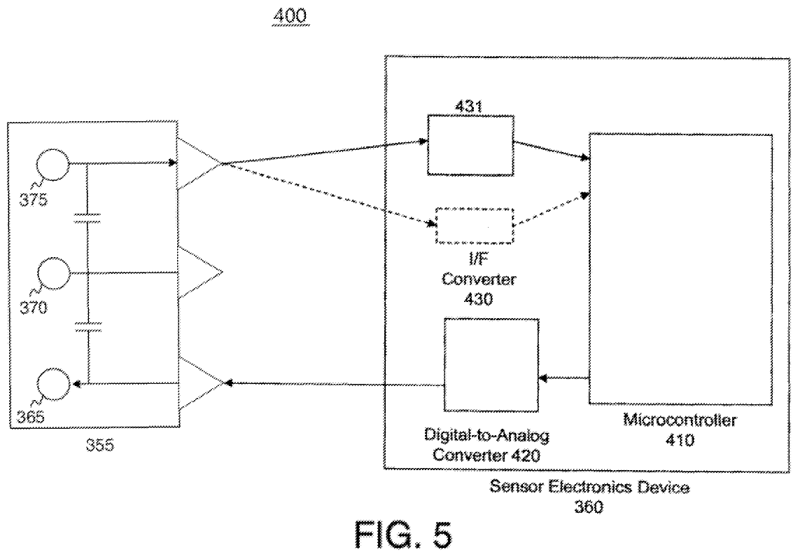

FIG. 5 illustrates an exemplary embodiment including a sensor and a sensor electronics device. The sensor set or sensor system 400 includes a sensor electronics device 360 and a sensor 355. The sensor includes a counter electrode 365, a reference electrode 370, and a working electrode 375. The sensor electronics device 360 includes a microcontroller 410 and a digital-to-analog converter (DAC) 420. The sensor electronics device 360 may also include a current-to-frequency converter (I/F converter) 430.

The microcontroller 410 includes software program code, which when executed, or programmable logic which, causes the microcontroller 410 to transmit a signal to the DAC 420, where the signal is representative of a voltage level or value that is to be applied to the sensor 355. The DAC 420 receives the signal and generates the voltage value at the level instructed by the microcontroller 410. In exemplary embodiments, the microcontroller 410 may change the representation of the voltage level in the signal frequently or infrequently. Illustratively, the signal from the microcontroller 410 may instruct the DAC 420 to apply a first voltage value for one second and a second voltage value for two seconds.

The sensor 355 may receive the voltage level or value. In an embodiment, the counter electrode 365 may receive the output of an operational amplifier which has as inputs the reference voltage and the voltage value from the DAC 420. The application of the voltage level causes the sensor 355 to create a sensor signal indicative of a concentration of a physiological characteristic being measured. In an embodiment, the microcontroller 410 may measure the sensor signal (e.g., a current value) from the working electrode. Illustratively, a sensor signal measurement circuit 431 may measure the sensor signal. In an embodiment, the sensor signal measurement circuit 431 may include a resistor and the current may be passed through the resistor to measure the value of the sensor signal. In an embodiment, the sensor signal may be a current level signal and the sensor signal measurement circuit 431 may be a current-to-frequency (I/F) converter 430. The current-to-frequency converter 430 may measure the sensor signal in terms of a current reading, convert it to a frequency-based sensor signal, and transmit the frequency-based sensor signal to the microcontroller 410. In exemplary embodiments, the microcontroller 410 may be able to receive frequency-based sensor signals easier than non-frequency-based sensor signals. The microcontroller 410 receives the sensor signal, whether frequency-based or non frequency-based, and determines a value for the physiological characteristic of a subject, such as a blood glucose level. The microcontroller 410 may include program code, which when executed or run, is able to receive the sensor signal and convert the sensor signal to a physiological characteristic value. In an embodiment, the microcontroller 410 may convert the sensor signal to a blood glucose level. In an embodiment, the microcontroller 410 may utilize measurements stored within an internal memory in order to determine the blood glucose level of the subject. In an embodiment, the microcontroller 410 may utilize measurements stored within a memory external to the microcontroller 410 to assist in determining the blood glucose level of the subject.

After the physiological characteristic value is determined by the microcontroller 410, the microcontroller 410 may store measurements of the physiological characteristic values for a number of time periods. For example, a blood glucose value may be sent to the microcontroller 410 from the sensor every second or five seconds, and the microcontroller may save sensor measurements for five minutes or ten minutes of BG readings. The microcontroller 410 may transfer the measurements of the physiological characteristic values to a display on the sensor electronics device 360. For example, the sensor electronics device 360 may be a monitor which includes a display that provides a blood glucose reading for a subject. In an embodiment, the microcontroller 410 may transfer the measurements of the physiological characteristic values to an output interface of the microcontroller 410. The output interface of the microcontroller 410 may transfer the measurements of the physiological characteristic values, e.g., blood glucose values, to an external device, e.g., an infusion pump, a combined infusion pump/glucose meter, a computer, a personal digital assistant, a pager, a network appliance, a server, a cellular phone, or any computing device.

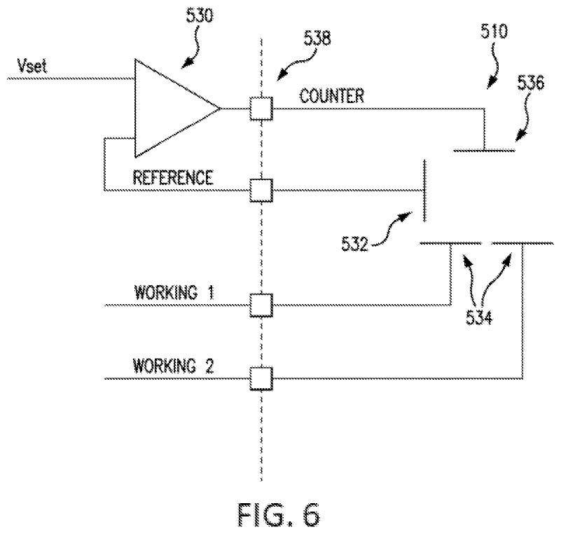

FIG. 6 illustrates an electronic block diagram of the sensor electrodes and a voltage being applied to the sensor electrodes according to an embodiment. In the embodiment illustrated in FIG. 6, an operational amplifier (op-amp) 530 or other servo controlled device may connect to sensor electrodes 510 through a circuit/electrode interface 538. The op amp 530, utilizing feedback through the sensor electrodes, attempts to maintain a prescribed voltage (what the DAC may desire the applied voltage to be) between a reference electrode 532 and a working electrode 534 by adjusting the voltage at a counter electrode 536. Current may then flow from a counter electrode 536 to a working electrode 534. Such current may be measured to ascertain the electrochemical reaction between the sensor electrodes 510 and the biomolecule of a sensor that has been placed in the vicinity of the sensor electrodes 510 and used as a catalyzing agent. The circuitry disclosed in FIG. 6 may be utilized in a long-term or implantable sensor or may be utilized in a short-term or subcutaneous sensor.

After forming the circuit from the electrodes exposed to the interstitial fluid, or other body fluid, an appropriate voltage is supplied across the working electrode and the reference electrode, such that the interstitial fluid provides impedance (R1 and R2) between the electrodes. An analog current signal Isig flows from the working electrode through the body (R1 and R2, which sum to Rs) and to the counter electrode. The voltage at the working electrode WRK is generally held to ground, and the voltage at the reference electrode may be based on half cell potential of the material used (i.e. 220 mV for Ag/AgCl reference electrode). Vset is generally from 300 to 700 mV, such as about 535 mV.

The most prominent reaction stimulated by the voltage difference between the electrodes is the reduction of glucose as it first reacts with GOX to generate gluconic acid and hydrogen peroxide (H₂O₂). Then the H₂O₂ is oxidized to hydrogen ions (2H⁺), electrons (2e⁻), and oxygen (O2) at the surface of the working electrode. The electron (2e−) charges traverse through the sensor conductive traces, hence causing an electrical current to flow. This results in the analog current signal (Isig) being proportional to the concentration of glucose in the interstitial fluid. The analog current signal (Isig) flows from the working electrode, to the counter electrode, typically through a filter and back to the low rail of the op-amp. An input to the op-amp is the set voltage Vset. The output of the op-amp adjusts the counter voltage Vctr at the counter electrode as Isig changes with glucose concentration. The voltage at the working electrode is generally held to ground, the voltage at the reference electrode is based on half cell potential of the material (i.e. 220 mV for Ag/AgCl reference electrode), and the voltage Vctr at the counter electrode varies as needed. In exemplary embodiments, more than one sensor may be used to measure blood glucose. In exemplary embodiments, redundant sensors may be used.

When a glucose oxidase (GOx) enzyme is used as a catalytic agent in a sensor, the flow of current from the counter electrode 536 to a working electrode 534 may be affected by the presence of an interferent or interferents in the body fluid, such as acetaminophen.

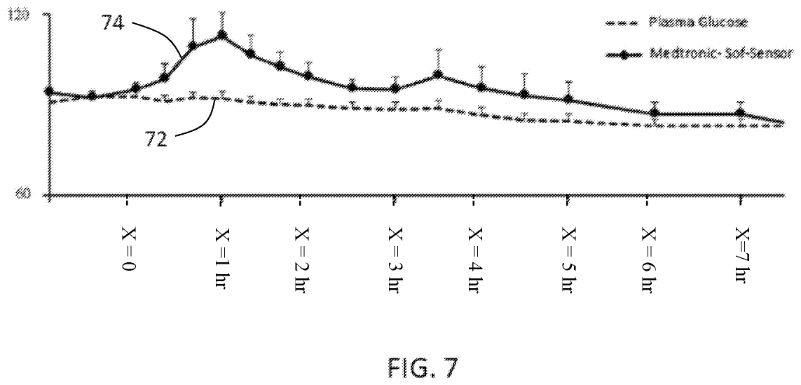

FIG. 7 illustrates the effect of an interferent, such as acetaminophen, on a glucose measurement for an exemplary glucose sensor. In FIG. 7, the x-axis represents time and the y-axis represents glucose concentration. The actual plasma glucose level is indicated by the line 72, while the measured sensor glucose level is indicated by line 74. In FIG. 7, 1 gram of acetaminophen is administered to the user at an initial time (X=0). As can be seen, the interferent affects the measured sensor glucose level, with a peak effect about one hour after administration and with effects lasting about seven hours.

FIG. 8 further illustrates acetaminophen measured in concentration in vivo in plasma, at line 82, and in interstitial fluid, at line 84. While the measured acetaminophen concentration in interstitial fluid is less than that of plasma, both are present for over six hours.

As described herein, sensor diagnostics are provided to detect the presence and/or concentration of an interferent, such as acetaminophen, and may be used to model an expected bias on glucose measurements due to the presence of the interferent, as suggested by FIGS. 7 and 8. Sensor diagnostics include the use of additional (diagnostic) information which can provide a real-time insight into the effect of the interferent on glucose measurements. In this regard, it has been discovered that Electrochemical Impedance Spectroscopy (EIS), an electrochemical technique to measure the impedance of a system in dependence of the input frequency, provides such additional information in the form of sensor impedance and impedance-related parameters at different frequencies. Aspects of the use of Electrochemical Impedance Spectroscopy (EIS) in Continuous Glucose Monitoring are described in detail in U.S. Pat. Nos. 10,660,555; 10,638,947; 10,342,468; 10,335,077; 10,335,076; 10,321,865; 10,321,844; 10,172,544; 10,156,543; 9,989,491; 9,989,490; 9,970,893; 9,863,911; 9,861,746; 9,808,191; 9,801,576; 9,649,059; 9,649,058; 9,645,111; 9,632,060; 9,625,415; 9,625,414; 9,408,567; 9,357,958; 9,213,010; 8,114,268; and 7,985,330; which are assigned to Medtronic, and which are incorporated herein by reference.

In exemplary embodiments, the EIS-based parameter of interest is the imaginary component of the impedance (Zimag), which may be obtained based on measurements of the impedance magnitude (|Z|) in ohms and the phase angle (Φ) in degrees of the electrode immersed in an electrolyte as is known. In particular, the imaginary component of the impedance is obtained at low frequencies.

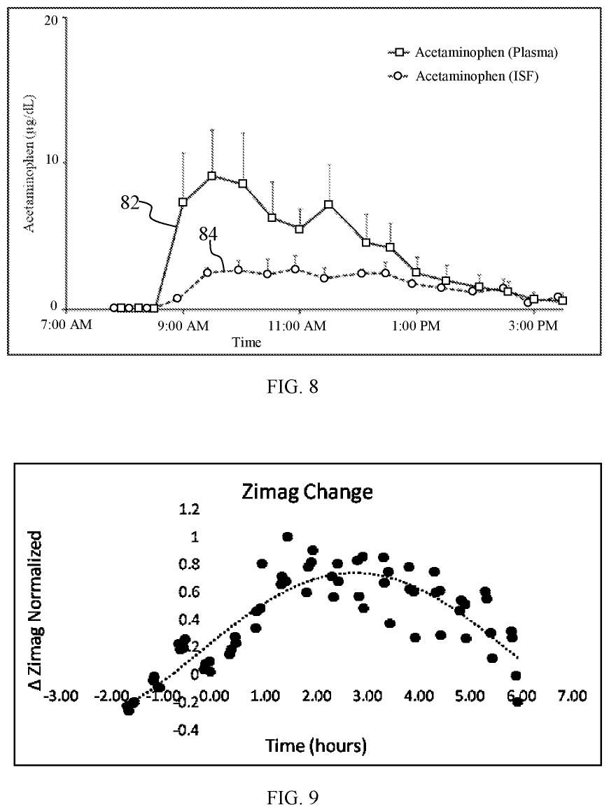

For example, FIG. 9 illustrates low frequency impedance sensing in vivo where acetaminophen is administered to the test subjects at time 0.00.

As shown, the impendence sensing profile follows pharmacokinetics of acetaminophen in vivo from plasma and interstitial fluid dialysate measurements.

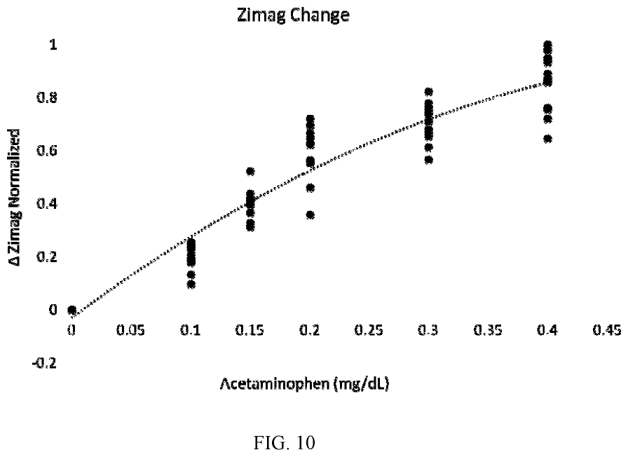

FIG. 10 presents data illustrating the effect of interferent concentration, in this case acetaminophen concentration, on the change in the imaginary component of impedance in an in vitro study. FIG. 10 illustrates acetaminophen dosing of 0.1, 0.15, 0.2, 0.3, and 0.4 mg/dL.

It has been found that frequencies sensitive to an interferent, such as acetaminophen, are low, for example not greater than 512 Hz, such as from 0.1 to 512 Hz. For example, such frequencies may include 0.4 Hz, 1.6 Hz, 2.5 Hz and 512 Hz, though other suitable frequencies may be used.

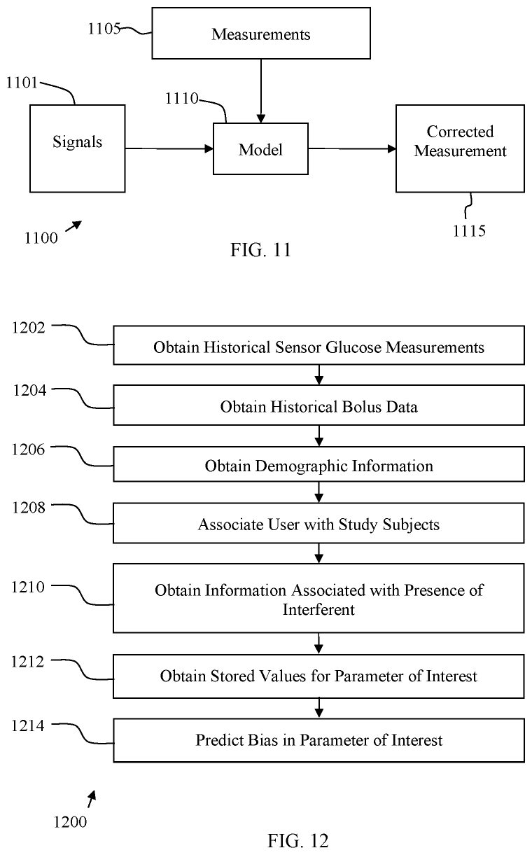

FIG. 11 illustrates a system 1100 for correcting sensor glucose measurements based on a modeled response to the presence of an interferent as suggested by FIGS. 7-10. In FIG. 11, the system 1100 includes a model 1110. Signals 1101 are input to the model 1110 as shown. Exemplary signals may include analog current signal (Isig), counter voltage (Vctr), and the change in the imaginary component of impedance (ΔZimag). Further, measurements 1105 may be input to the model 1110. Measurements 1105 may include sensor glucose measurements, and amount of interferent administered. In certain embodiments, the amount of interferent administered may be input by a user. In certain embodiments, the amount of interferents administered may be determined by analysis of other measurements and/or signals.

In some examples, model 1110 may be implemented as one or more artificial neural networks, genetic programming, support vector machines, Bayesian networks, probabilistic machine learning models, or other Bayesian techniques, fuzzy logic, heuristically derived combinations, or the like. The model 1110 may operate according to the exemplary modeling process 1200 of FIG. 12 to provide a modeled measurement indicative of the biased measurement due to the presence of the interferent in the monitored body fluid. Then, the system 1100 may provide a corrected measurement 1115.

FIG. 12 depicts an exemplary modeling process 1200 suitable for implementation by the system 1100. The various tasks performed in connection with the modeling process 1200 may be performed by hardware, firmware, software executed by processing circuitry, or any combination thereof. In practice, portions of the modeling process 1200 may be performed by different elements of the patient management system 1100, such as, for example, an infusion device, a sensing arrangement, a server, a database, a client device or application, and/or a processing system. It should be appreciated that the modeling process 1200 may include any number of additional or alternative tasks, the tasks need not be performed in the illustrated order and/or the tasks may be performed concurrently, and/or the modeling process 1200 may be incorporated into a more comprehensive procedure or process having additional functionality not described in detail herein. Moreover, one or more of the tasks shown and described in the context of FIG. 12 could be omitted from a practical embodiment of the modeling process 1200 as long as the intended overall functionality remains intact.

The modeling process 1200 may include obtaining historical measurement data for the user of interest and obtaining historical bolus data for the patient over a period of time (e.g., a month, several months, a year, or any other period of time) corresponding to the historical measurement data (tasks 1202, 1204). For example, the infusion device may periodically upload, to a server via a network, reference blood glucose measurement values obtained from the body of the patient (e.g., using a blood glucose meter or fingerstick device) along with bolus information including the timings and amounts of insulin delivered, including indications of whether a particular bolus is a meal bolus or otherwise associated with a meal. In other examples, the user may manually enter reference blood glucose measurement values and/or bolus information via a computer, smartphone, or any other electronic device. The bolus information may also include the amount of carbohydrates consumed, the type of meal, or the like. In this regard, in the absence of an explicit meal indication or announcement from the patient, the server may automatically classify a bolus delivered as a meal bolus when a carbohydrate entry occurred within a threshold amount of time of the bolus being delivered (e.g., within 5 minutes). Additionally, the infusion device (or alternatively, the sensing arrangement) may periodically upload, to the server, sensor glucose measurement values obtained from the body of the patient by the sensing arrangement. In exemplary embodiments, the historical measurement values may also be stored in association with a current location of the sensing arrangement (or sensor site location) on the body of the patient at the time the respective measurement values were obtained. Further, the user may manually enter a declaration that the user ingested acetaminophen or another interferent via computer, smartphone, or other electronic device. Additionally, the user may manually enter the amount of interferent ingested via computer, smartphone, or other electronic device.

In addition, the historical measurement data and historical bolus data may include data for other subjects. Thus, the modeling process 1200 may include obtaining historical data and historical bolus data for other subjects at tasks 1202 and 1204.

The illustrated modeling process 1200 also obtains demographic information or other clinically-relevant information associated with the user (task 1206). Demographic information associated with the patient may be input or otherwise provided by the user and uploaded to a server for storage in a database in association with the user. The demographic information may include, for example, the patient's height, weight, body mass index, age, ethnicity, residence information, or other information that may be utilized to classify the patient. In this regard, as demographic information associated with the patient changes (e.g., the patient gains or loses weight, ages, relocates, etc.), such updated demographic information may be uploaded or otherwise provided to the server to update the patient's history stored in the database. The demographic information may also be stored in association with a timestamp or other temporal information to facilitate analysis and establishing correlations with other data to generate patient-specific models, as described below. Other clinically-relevant information may be obtained and utilized, either in addition to or alternatively to the demographic information. Such clinically-relevant information may include, for example, the patient's medical history, the patient's medication or drug history, the patient's hospitalization or other treatment information and records, or the like. For purposes of explanation the subject matter is primarily described herein in the context of utilizing demographic information, but it should be appreciated that clinically-relevant information may be similarly utilized in an equivalent manner.

In exemplary embodiments, the user may be assigned or otherwise associated with a particular group of study subjects having one or more characteristics in common based on the demographic information associated with that patient, with a parameter model for that study group being determined based on the aggregated historical data for the different subjects of the group (task 1208). In exemplary embodiments, the modeling process 1200 assigns or otherwise associates the user with a study group parameter model upon initialization of the user within the system 1100. It is further noted that the associated study subjects may be considered during the performance of steps 1202 and 1204 such that historical measurement data and historical bolus data may be obtained for the associated study subjects as well as for the user.

In exemplary embodiments, the modeling process 1200 continues by obtaining information associated with the presence of an interferent in the body fluid of the patient (task 1210). In this regard, environmental and behavioral information concurrent to the measurement data, the delivery data, or potentially other data may be obtained and used to facilitate analysis of relationships between such data.

As noted above, in certain embodiments, the information associated with the presence of an interferent in the body fluid of the patient, i.e., indication that interferent was ingested, indication that interferent was ingested and time of ingestion and/or size of dose, is input by the user into an infusion device, other client device, or through a computer, smartphone or other device. In certain embodiments, the information associated with the presence of an interferent in the body fluid is ascertained through analysis of relevant signals such as analog current signal (Isig), counter voltage (Vctr), or the change in the imaginary component of impedance (ΔZimag) or through sensor glucose (SG) rate of change.

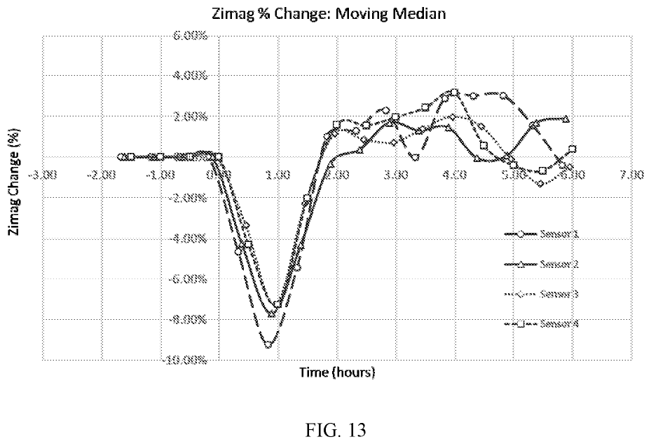

In an exemplary embodiment, Zimag % change is monitored to determine that an interferent is present in the body fluid of the patient. While various methods of using Zimag % change are possible, one method using a moving median window can be understood in relation to FIG. 13. FIG. 13 illustrates a percent change in the imaginary component of impedance (i.e., relative to a normalized Zimag) at four sensors for one study subject in response to the presence of an interferent. EIS signals were monitored for each sensor. While FIG. 13 indicates that EIS signal measurements were taken every 30 minutes, such measurements can be taken at any desired interval, such as for example at every 5, 10, 15, 20 or 30 minutes, or at longer intervals. In FIG. 13, at Time=0, an interferent, e.g., acetaminophen, was ingested by the study subject. For each sensor, Zimag % change was calculated by comparing a newly obtained Zimag value to a median Zimag value from a preceding period of time. In exemplary embodiments, the preceding period of time may occur over one hour, 90 minutes, two hours, three hours, or other desired period.

For example, for a measurement interval of 30 minutes and a comparison period of time of two hours for Sensor 4, the signal obtained at a measurement time, e.g., at Time=30 minutes, is compared to the median of the signals taken for sensor 1 at Time=0, Time=−30 minutes, Time=−1 hour, and Time=−90 minutes. In FIG. 13, the resulting Zimag % change value for Sensor 4 is about −4.3%.

The method may include identifying a threshold Zimag % change value that is indicative of the presence of an interferent in the body fluid. The threshold Zimag % change value may be identified based on historical measurement data and historical bolus data, and/or on in vitro data. Thus, for the study subject of FIG. 13, if the threshold Zimag % change value is identified as −6.0%, then each sensor would indicate the presence of an interferent before Time=1 hour.

As noted above, an exemplary embodiment may alternatively or additionally provide an indication of the presence of an interferent based on the sensor glucose (SG) signal rate of change. For example, the SG signal rate of change may be calculated based on the equation:

SG_{ROC}=(SGₜ₂−SGₜ₁)/(t2−t1)

Wherein a preceding or first SG signal (SGₜ₁) is taken at time t1 and a current or second SG signal (SGₜ₂) is taken at time t2. In an exemplary embodiment, the difference in time between t1 and t2 is 5 minutes (i.e., where t1=5 min, and t2=10 min), which would correspond to consecutive SG measurements during a typical operation where SG is sampled every 5 minutes. Alternatively, the difference between t1 and t2 can be 1, 2, 10, 15, or 20 minutes, or other desired interval.

Referring back to FIG. 12, it is noted that the information associated with the presence of an interferent in the body fluid of the patient may also be stored in association with a timestamp or other temporal information to facilitate analysis and establishing correlations with other data for generating patient-specific models.

In exemplary embodiments, the modeling process 1200 also obtains stored values for the parameter of interest to be modeled for the patient (task 1212). In this regard, the historical values for the parameter being modeled may be stored in a library that may be uploaded to a server via a network or otherwise available to the modeling process 1200.

In one or more embodiments, the server stores or otherwise maintains, in the database, one or more files or entries associated with the patient that maintains an association between the patient's historical sensor glucose measurement data, the patient's historical bolus and meal data, the patient's historical reference blood glucose measurements, the patient's current and/or past demographic information, the historical context information associated with operation of the patient's sensing arrangement and/or infusion device (e.g., historical environmental data, behavioral data, and the like), and historical values for the parameter of interest, along with timestamps or other temporal information associated with the respective pieces of historical data. It should be noted that the modeling process 1200 may support modeling any number of parameters of interest, such that the database may store historical values for any number of parameters or variables utilized by the sensing arrangement and/or the infusion device to support respective operation thereof. In this regard, one parameter of interest may be modeled as a function of other parameters or variables in addition to the historical measurement, delivery, and contextual data, and those parameters or variables themselves may also be modeled as a function of the historical measurement, delivery, and contextual data.

The modeling process 1200 continues with utilizing the information associated with the presence of an interferent in the body fluid of the patient in the study group parameter model to predict a bias in the parameter of interest over a selected time interval (task 1214).

In exemplary embodiments, the model utilizes machine learning to determine which combination of historical sensor measurement data, historical delivery data, demographics data, environmental data, behavioral data, and other historical parameter data are most strongly correlated to or predictive of the contemporaneous historical values for the parameter of interest, and then determines a corresponding equation for calculating the bias value of the parameter of interest based on that subset of input variables. Thus, the model is capable of characterizing or mapping a particular combination of one or more of the current (or recent) sensor glucose measurement data, delivery data, demographic information, environmental conditions, patient behavior, and the like to a current value for the parameter of interest, and vice versa. Since each patient's physiological response may vary from the rest of the population, the subset of input variables that are predictive of or correlative to the parameter of interest for that patient may vary from other users. Additionally, the relative weightings applied to the respective variables of that predictive subset may also vary from other patients who may have common predictive subsets, based on differing correlations between a particular input variable and the historical values of the parameter of interest for that particular patient. It should be noted that any number of different machine learning techniques may be utilized by the server to determine what input variables are predictive of the parameter of interest for the current patient of interest, such as, for example, artificial neural networks, genetic programming, support vector machines, Bayesian networks, probabilistic machine learning models, or other Bayesian techniques, fuzzy logic, heuristically derived combinations, or the like.

In one or more exemplary embodiments, only a subset of the historical data for the patient are used to select the parameter model, with the remaining historical data being utilized by the modeling process 1200 to test or otherwise validate the selected model. For example, for the testing subset of the historical data, the server applies the selected parameter model to the predictive variable values contemporaneous to or otherwise temporally associated with historical values for the modeled parameter, and then identifies or otherwise determines whether the model results are correlative to those historical values for the modeled parameter. In this regard, the server compares the model-based parameter value calculated based on the predictive subset of historical data to the corresponding historical values for the modeled parameter and calculates or otherwise determines one or more metrics indicative of the performance of the model. For example, the server may calculate or otherwise determine one or more correlation coefficient values associated with the developed model based on the differences between the model-based calculated parameter values and the corresponding historical values for the modeled parameter.

It should be noted that in one or more embodiments, the modeling process 1200 may be performed repeatedly to dynamically update the model(s) substantially in real-time. For example, whenever new data becomes available from a particular source within the system, the modeling process 1200 may be repeated to dynamically update the parameter model as appropriate. That said, in other embodiments, once a sufficient amount of data has been obtained, or the parameter model has stabilized (e.g., no changes over a certain number of successive iterations of the modeling process 1200), the parameter model may be persisted and the modeling process 1200 may not be continually performed.

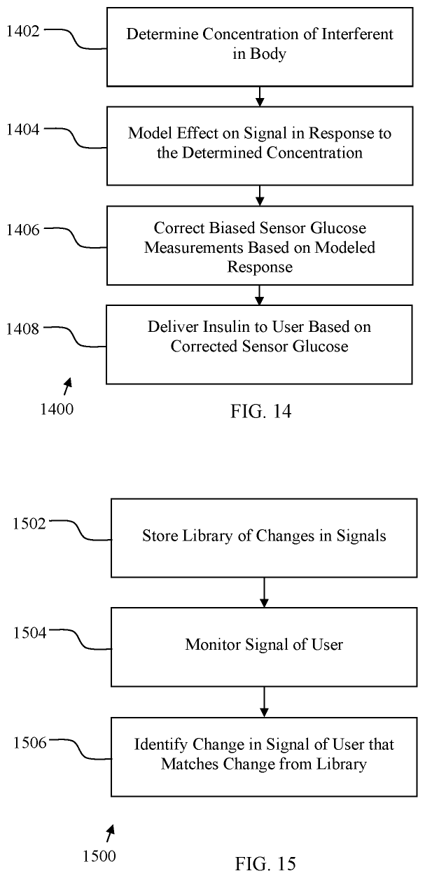

FIG. 14 depicts an exemplary method 1400 for operating a sensing device associated with a user. As shown, method 1400 includes determining a concentration of interferent in a body (task 1402). As described above, task 1302 may be performed by receiving an input from the user indicating the amount of interferent consumed or otherwise administered. Further, task 1402 may be performed by analyzing signals, such as EIS signals, and determining the concentration of interferent in the body based on comparison of signal changes over time to a library of data associated with known interferent concentrations.

Method 1400 further includes modeling an effect on a signal, such as an Isig signal, in response to the determined concentration of the interferent (task 1404). As described above, such modeling may include identification of, and association with, a prior-studied group of subjects sharing selected characteristics with the user. Further, such modeling may include use of data from the selected subject group. As a result, an effect on the signal over time may be identified, such as a bias on a selected signal or parameter. Further, it is noted that the modeling may be utilized for any interferent being detected, or may be specific to the interferent being detected. In other words, a single model may be used for every interferent or a plurality of models may be used, with each model dedicated to a specific interferent or to interferents with common characteristic signal responses.

Method 1400 may also include correcting a biased sensor analyte, such as sensor glucose measurements, based on the modeled response (task 1406). Specifically, the sensor glucose measurement may be adjusted in view of the modeled response to create a corrected sensor glucose value. In exemplary embodiments, the sensor glucose measurement is adjusted by applying the model to the input parameters to produce the corrected sensor glucose value.

Thereafter, the method 1400 may continue with delivery of insulin to the user based on the corrected sensor glucose value (i.e., not based on the sensor glucose measurement) (task 1408).

FIG. 15 illustrates an exemplary method 1500 for determining a concentration of interferent, such as acetaminophen, in a body. As shown, method 1500 includes storing a library of data including signals for a study of subjects having various concentrations of the interferent in body fluids over time (task 1502). In particular, changes in EIS signals over time may be of use.

Method 1500 further includes monitoring signals of the user (task 1504). In particular, the analog current signal (Isig), counter voltage (Vctr), the change in the imaginary component of impedance (ΔZimag), and/or the SG signal rate of change may be measured.

Method 1500 also includes identifying a change in a signal that matches a change in the data stored in the library that is indicative of the presence of an interferent (task 1506). In particular, changes in the imaginary component of impedance signal may be compared with the library of imaginary component of impedance signals and analyzed to determine if an interferent is present in the body fluid, and if so, the concentration of interferent in the body fluid. Such a comparison may be performed with in vitro data. For example, a library of Isig signals recorded in response to known acetaminophen concentrations may be inputs and can be used to calculate the acetaminophen concentration from an in vivo signal response.

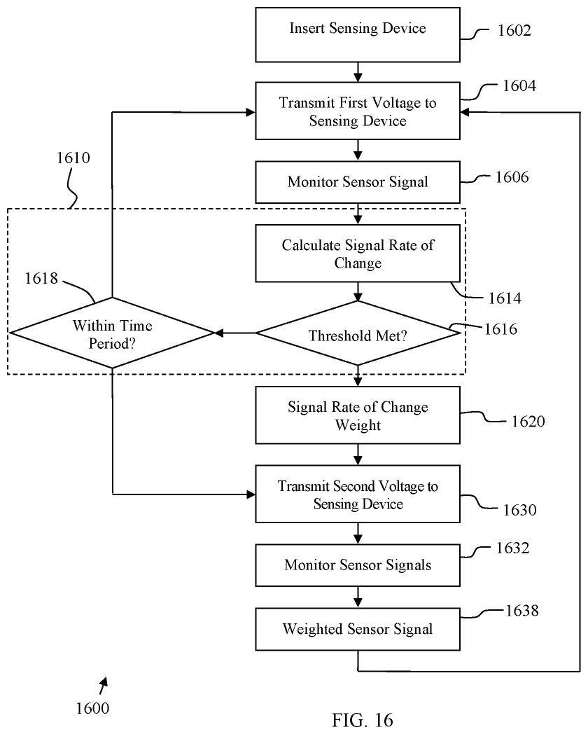

FIG. 16 provides a flow chart for an exemplary embodiment of a method for operating a sensing device associated with a user, wherein the sensing device includes a controller coupled to a sensing element configured to measure a physiological condition in a body of the user. As shown, method 1600 may include insert the sensing device into contact with the body fluid and connecting the sensing device, transmitter, and/or other components (task 1602). The method further includes transmitting a first voltage to the sensing device at task 1604. The first voltage may be considered to be the set voltage (Vₛₑₜ). An exemplary first voltage is from 300 to 700 mV, such as 535 mV. The method includes monitoring a first sensor signal from the sensing device in response to the first voltage at task 1606. For example, the first sensor signal may be the sensor glucose (SG) signal as described above.

Monitoring the first sensor signal from the sensing device in response to the first voltage may comprise taking a plurality of measurements of the first sensor signal over a defined time period.

Method 1600 further includes learning, with the controller, that an interferent is in a body fluid at task 1610. In exemplary embodiments, learning that the interferent is in the body fluid is passive and relies on receiving inputted information from the user. If no information is inputted from the user, then the method 1600 continues with transmitting the first voltage to the sensing device at task 1604. If, however, the user inputs information that an interferent was ingested, then the method 1600 continues at task 1620 as described below.

In exemplary embodiments, and as illustrated, learning that an interferent is in the body fluid (1610) is active and includes determining that the interferent is in the body fluid at task 1614 and query 1616. In some embodiments, determining that the interferent is in the body fluid includes determining a concentration of the interferent in the body fluid. As shown, at task 1614 a first sensor signal rate of change is calculated with the controller. Calculating the first sensor signal rate of change may comprise determining the slope of the plurality of measurements of the first sensor signal over a defined time period. At query 1616 it is identified whether the first sensor signal rate of change is greater than a threshold indicative of the presence of the interferent in the body fluid.

Method 1600 may end with a positive identification of the presence of the interferent in the body fluid such that method 1600 includes only the identification that the interferent is in the body fluid. Additional actions may be taken in response to such identification. For example, as described above, the signal data obtained may be fed into a model to provide a corrected sensor signal as described above.

In the illustrated method 1600 of FIG. 16, if the threshold of query 1616 is not met, then the method further assesses at query 1618 if there was an interferent detection event within a preceding selected time period, such as within the last 6.5 hours. If no event has been detected within the preceding selected time period, then the method continues by returning to task 1604 and transmitting the first voltage to the sensing device. In other words, after detecting the absence of the interferent in the body fluid, the method 1600 repeats transmitting the first voltage to the sensing device at task 1604 and monitoring the sensor signal from the sensing device in response to the first voltage at task 1606. If query 1618 determines that there was an interferent detection event within the preceding selected time period, then the method continues at task 1630 as described below.

Returning to query 1616, if the threshold of query 1616 is met, then the method 1600 continues with calculating a signal rate of change weight at task 1620. In an exemplary embodiment, the signal rate of change weight is calculated by dividing the rate of change signal before reaching the threshold at query 1616 by the peak rate of change after exceeding the threshold at query 1616. This signal rate of change weight calculated from task 1620 is then stored for subsequent use. The method 1600 then continues at task 1630.

At task 1630, the method 1600 includes transmitting a second voltage to the sensing device. In exemplary embodiments, the second voltage is less than the first voltage. For example, the second voltage may be from about 40 to about 450 mV. Within task 1630, the second lower voltage may consist of a constant voltage or alternating voltage. In an exemplary embodiment, the second lower voltage is alternated at frequency of 0.105 Hz.

The method 1600 continues at task 1632 by monitoring sensor signals from the sensing device in response to the second voltage. An exemplary sensor signal is the sensor glucose (SG) signal as described above. Within task 1632, in an exemplary embodiment of an alternating second voltage at 0.105 Hz frequency, an impedance magnitude may be determined and corresponding sensor current (Isig) signal calculated per the second lower voltage in task 1630. Subsequently, a sensor glucose calibration ratio for the second lower voltage may be determined by dividing the sensor glucose value from the first voltage by the sensor current (Isig) signal calculated per the second lower voltage at task 1638 based on the equation:

$$SG {CR}_{V 2} = \frac{{SG}_{V 1}}{{Isig}_{V 2}}$$

The method 1600 then continues by calculating a weighted sensor glucose calibration ratio at task 1638 by using the sensor glucose calibration ratio from voltage 2 and the rate of change weight from task 1620 per the equation below:

SG CR_{w}=SG CR_{V2}−(SG CR_{V2}×ROC Weight)

The weighted sensor glucose calibration is then multiplied by the sensor current (Isig) calculated per the second lower voltage in task 1630 per the equation below:

SG_{corr}=SG CR_{w}×Isig_{V2}

As described above, the weighted SG signal in response to the signal rate of change and lower second voltage may more accurately represent the actual glucose level in the body fluid. The weighted sensor signal at task 1638 may be used by the controller as a corrected signal in replacement of the sensor signal monitored at task 1606, such that exogenous error is reduced.

After transmitting the lower second voltage to the sensing device, monitoring signals in response to the second voltage, and calculating the weighted sensor signal, the method 1600 may continue by transmitting the higher first voltage to the sensing device at task 1604. And repeating the method 1600 beginning at task 1606.

As can be seen, when the threshold at query 1616 is met, i.e., an interferent is detected, at successive iterations the method 1600 will include alternating application of the first voltage and the second voltage to the sensing device and monitoring the sensor signal in response to the first voltage and the sensor signal in response to the second voltage until a time of the selected duration, such as 6.5 hours from the detection event, has elapsed. As an interferent such as acetaminophen may be present in body for hours, the alternating application of the two voltages may occur for hours. In an exemplary embodiment, alternating application of the first voltage and the second voltage to the sensing device comprises transmitting the first voltage to the sensing device for a first period of from about 1 to about 30 minutes and transmitting the second voltage to the sensing device for a second period of from about 1 to about 30 minutes. It is contemplated that the first period may be of any suitable duration and the second period may be of any suitable duration.

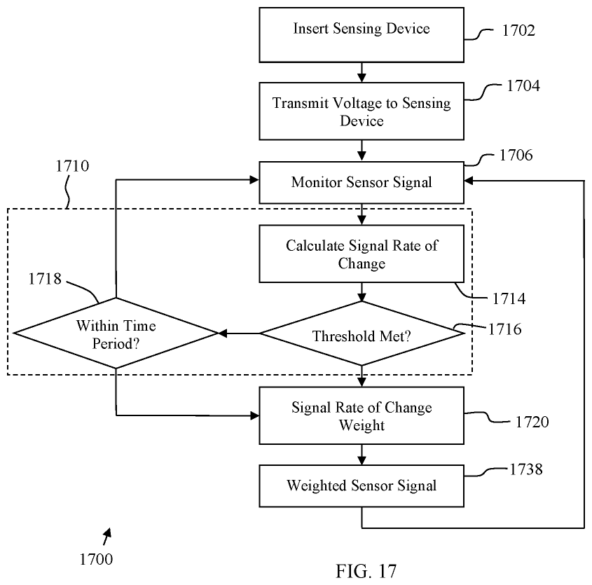

FIG. 17 provides a flow chart for another exemplary embodiment of a method for operating a sensing device associated with a user, wherein the sensing device includes a controller coupled to a sensing element configured to measure a physiological condition in a body of the user. The method 1700 of FIG. 17 additionally uses a single voltage for the “weighted sensor signal” for signal correction per a method similar to that of method 1600.

As shown, method 1700 may include insert the sensing device into contact with the body fluid and connecting the sensing device, transmitter, and/or other components (task 1702). The method further includes transmitting a voltage to the sensing device at task 1704. The voltage may be considered to be the set voltage (Vₛₑₜ). An exemplary voltage is from 300 to 700 mV, such as 535 mV. The method includes monitoring a sensor signal from the sensing device in response to the voltage at task 1706. For example, the sensor signal may be the sensor glucose (SG) signal as described above.

Monitoring the sensor signal from the sensing device in response to the voltage may comprise taking a plurality of measurements of the sensor signal over a defined time period.

Method 1700 further includes learning, with the controller, that an interferent is in a body fluid at task 1710. In exemplary embodiments, learning that the interferent is in the body fluid is passive and relies on receiving inputted information from the user. If no information is inputted from the user, then the method 1700 continues with transmitting the voltage to the sensing device at task 1704. If, however, the user inputs information that an interferent was ingested, then the method 1700 continues at task 1720 as described below.

In exemplary embodiments, and as illustrated, learning that an interferent is in the body fluid (1710) is active and includes determining that the interferent is in the body fluid at task 1714 and query 1716. In some embodiments, determining that the interferent is in the body fluid includes determining a concentration of the interferent in the body fluid. As shown, at task 1714 a sensor signal rate of change is calculated with the controller. Calculating the sensor signal rate of change may comprise determining the slope of the plurality of measurements of the sensor signal over a defined time period. At query 1716 it is identified whether the sensor signal rate of change is greater than a threshold indicative of the presence of the interferent in the body fluid.

Method 1700 may end with a positive identification of the presence of the interferent in the body fluid such that method 1700 includes only the identification that the interferent is in the body fluid. Additional actions may be taken in response to such identification. For example, as described above, the signal data obtained may be fed into a model to provide a corrected sensor signal as described above.

In the illustrated method 1700 of FIG. 17, if the threshold of query 1716 is not met, then the method further assesses at query 1718 if there was an interferent detection event within a preceding selected time period, such as within the last 6.5 hours. If no event has been detected within the preceding selected time period, then the method continues by returning to task 1704 and transmitting the voltage to the sensing device. In other words, after detecting the absence of the interferent in the body fluid, the method 1700 repeats transmitting the voltage to the sensing device at task 1704 and monitoring the sensor signal from the sensing device in response to the voltage at task 1706. If query 1718 determines that there was an interferent detection event within the preceding selected time period, then the method continues at task 1720 as described below.

Returning to query 1716, if the threshold of query 1716 is met, then the method 1700 continues with calculating a signal rate of change weight at task 1720. In an exemplary embodiment, the signal rate of change weight is calculated by dividing the rate of change signal before reaching the threshold at query 1716 by the peak rate of change after exceeding the threshold at query 1716. The method 1700 then continues at task 1738 where a corrected sensor glucose signal, i.e., a weighted SG signal, may be determined by multiplying the sensor glucose value by the signal rate of change weight found in task 1720. The weighted SG signal may more accurately represent the actual glucose level in the body fluid. The weighted sensor signal at task 1738 may be used by the controller as a corrected signal in replacement of the sensor signal monitored at task 1706, such that exogenous error is reduced.

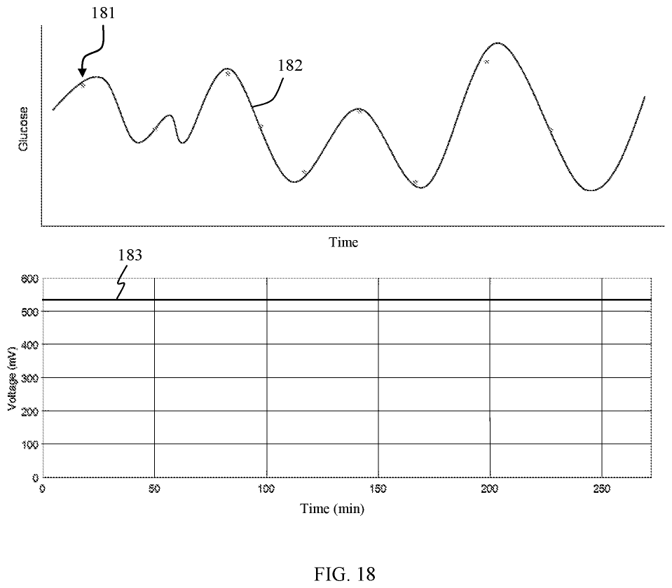

FIGS. 18-20 illustrate a hypothetical effect of an interferent on a sensor glucose signal as compared to accurate blood glucose measurements, and the correction provided by methods described herein. In the upper graph of FIG. 18, glucose in mg/dL is measured on the y-axis against time on the x-axis. In the lower graph of FIG. 18, voltage in mV is measured on the y-axis against time on the x-axis. As shown, nine blood glucose measurements are represented by points 181 while sensor glucose measurements form the line 182. As can be seen, there is good agreement between the blood glucose (BG) measurements and the sensor glucose (SG) measurements. During the time illustrated in the graph of FIG. 18, the presence of an interferent would not be indicated. Thus, in FIG. 18 and under embodiments herein, only a single voltage or first voltage 183 is applied to the sensing device. FIG. 18 illustrates that a voltage 183 of 535 mV is applied to the sensing device.

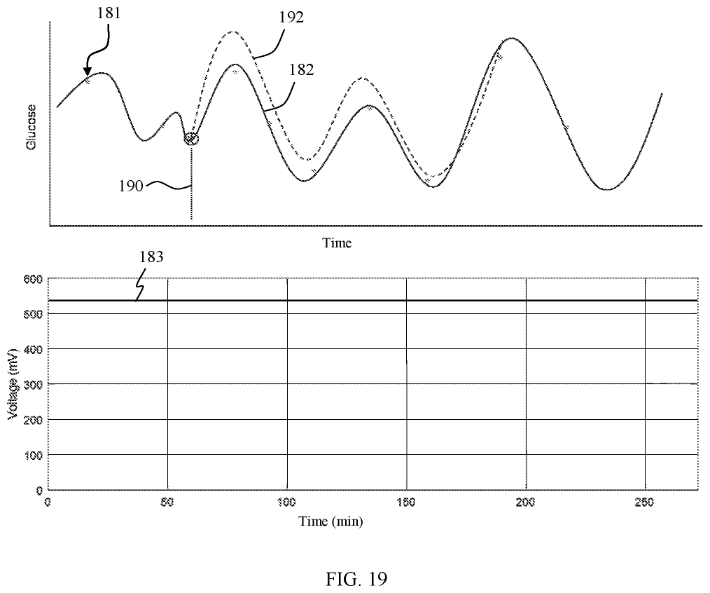

FIG. 19 provides the same graphs as FIG. 18 under a regime in which an interferent is introduced to the body fluid at time 190. As shown, the nine blood glucose measurements 181 remain the same as in FIG. 18. However, in FIG. 19, line 192 shows how the sensor glucose measurements are affected after ingestion of the interferent at time 190. Specifically, for a period of time after ingestion of the interferent, the sensor glucose measurements at line 192 are shifted away from the accurate line 182 representative of measurements taken without the presence of an interferent. Thus, in a regime as shown in FIG. 19 where the same first voltage 183 is applied to the sensing device, the sensor is not provided accurate sensor glucose measurements.

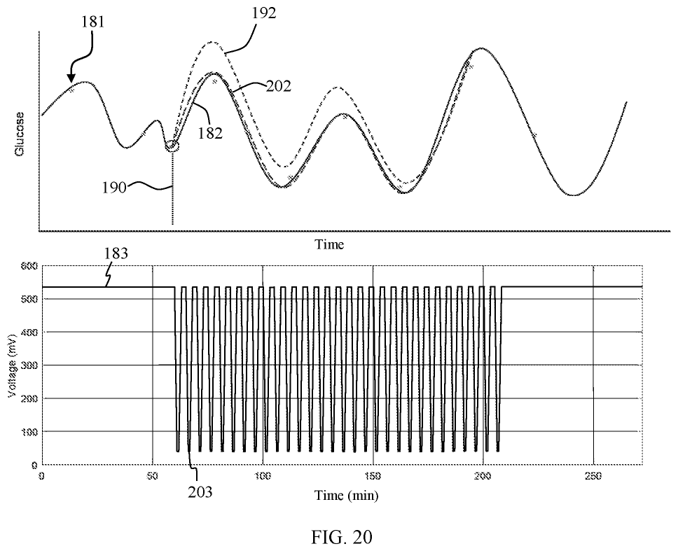

FIG. 20 provides the same graphs as FIGS. 18-19 under a regime in which an interferent is introduced to the body fluid at time 190 and in which a method for correcting the sensor glucose measurements is provided. The nine blood glucose measurements 181 remain the same as in FIGS. 18-19. Again, the correct sensor glucose measurements without the presence of an interferent are represented by line 182 and the incorrect sensor glucose measurements, taken at the higher first voltage 183 in the presence of an interferent as described in FIG. 19, is illustrated as line 192. In FIG. 20, however, upon the indication, such as a sensor glucose rate of change above the threshold, that an interferent is present, the voltage is pulse modulated such that a lower second voltage 203 is applied to the sensing device. In FIG. 20, thirty pulses of the lower second voltage 203 are transmitted to the sensing device. Further, the sensor glucose signal is measured in response to the second voltage 203 to form corrected sensor glucose measurements as indicated by line 202. As may be seen, the corrected line 202 is fit to the blood glucose measurements 181, indicating that the corrected sensor glucose measurements are accurate.

Unless specifically stated otherwise, as is apparent from the preceding discussion, it is to be appreciated that throughout this specification discussions utilizing terms such as “processing”, “computing”, “calculating”, “determining”, “estimating”, “selecting”, “identifying”, “obtaining”, “representing”, “receiving”, “transmitting”, “storing”, “analyzing”, “associating”, “measuring”, “detecting”, “controlling”, “delaying”, “initiating”, “setting”, “delivering”, “waiting”, “starting”, “providing”, and so forth may refer to actions, processes, etc. that may be partially or fully performed by a specific apparatus, such as a special purpose computer, special purpose computing apparatus, a similar special purpose electronic computing device, and so forth, just to name a few examples. In the context of this specification, therefore, a special purpose computer or a similar special purpose electronic computing device or apparatus may be capable of manipulating or transforming signals, which are typically represented as physical electronic and/or magnetic quantities within memories, registers, or other information storage devices; transmission devices; display devices of a special purpose computer; or similar special purpose electronic computing device; and so forth, just to name a few examples. In particular embodiments, such a special purpose computer or similar may include one or more processors programmed with instructions to perform one or more specific functions. Accordingly, a special purpose computer may refer to a system or a device that includes an ability to process or store data in the form of signals. Further, unless specifically stated otherwise, a process or method as described herein, with reference to flow diagrams or otherwise, may also be executed or controlled, in whole or in part, by a special purpose computer.

It should be noted that although aspects of the above apparatuses, methods, sensors, devices, processes, etc. have been described in particular orders and in particular arrangements, such specific orders and arrangements are merely examples and claimed subject matter is not limited to the orders and arrangements as described. It should also be noted that systems, devices, methods, processes, etc. described herein may be capable of being performed by one or more computing platforms. In addition, instructions that are adapted to realize methods, processes, etc. that are described herein may be capable of being stored on a storage medium as one or more machine readable instructions. If executed, machine readable instructions may enable a computing platform to perform one or more actions. “Storage medium” as referred to herein may relate to media capable of storing information or instructions which may be operated on, or executed by, one or more machines (e.g., that include at least one processor). For example, a storage medium may include one or more storage articles and/or devices for storing machine-readable instructions or information. Such storage articles and/or devices may include any one of several non-transitory media types including, for example, magnetic, optical, semiconductor, a combination thereof, or other storage media. By way of further example, one or more computing platforms may be adapted to perform one or more processes, methods, etc. in accordance with claimed subject matter, such as methods, processes, etc. that are described herein. However, these are merely examples relating to a storage medium and a computing platform and claimed subject matter is not limited in these respects.

Although what are presently considered to be example features have been illustrated and described, it will be understood by those skilled in the art that various other modifications may be made, and equivalents may be substituted, without departing from claimed subject matter. Additionally, many modifications may be made to adapt a particular situation to the teachings of claimed subject matter without departing from central concepts that are described herein. Therefore, it is intended that claimed subject matter not be limited to particular examples disclosed, but that such claimed subject matter may also include all aspects falling within the scope of appended claims, and equivalents thereof.

What is claimed is:

1. A method for operating a sensing device associated with a user, the sensing device including a controller coupled to a sensing element configured to measure a physiological condition in a body of the user, the method comprising:
storing a library of changes in electrochemical impedance spectroscopy (EIS) signals correlated to known concentrations of an interferent within bodies of study subjects, wherein the library is accessible to the controller;
monitoring, by the controller, EIS signals of the user;
matching, by the controller, a change in an EIS signal of the user with a change in a selected EIS signal from the library; and
determining a concentration of the interferent within the body of the user based on the selected EIS signal from the library.

2. The method of claim 1, wherein:
the library of changes in EIS signals is obtained at a selected frequency from about 0.1 to about 512 Hz; and
monitoring EIS signals of the user comprises monitoring EIS signals of the user at the same selected frequency.

3. The method of claim 1, wherein:
the library of changes in EIS signals correlated to known concentrations of the interferent within bodies of study subjects includes an imaginary component of the impedance signal (Zimag);
monitoring EIS signals of the user with the controller comprises monitoring the imaginary component of the impedance signal of the user;
matching the change in an EIS signal of the user with the change in the selected EIS signal from the library comprises matching a change in the imaginary component of the impedance signal of the user with a change in a selected imaginary component of the impedance signal from the library.

4. The method of claim 1, wherein:
the library of changes in EIS signals correlated to known concentrations of the interferent within bodies of study subjects includes a counter voltage signal;
monitoring EIS signals of the user with the controller comprises monitoring counter voltage signals of the user;
matching the change in an EIS signal of the user with a change in a selected EIS signal from the library comprises matching a change in the counter voltage signal of the user with a change in a selected counter voltage signal from the library.

5. The method of claim 1, further comprising modeling an effect on a selected EIS signal in response to the concentration of the interferent determined to be within the body of the user.

6. The method of claim 1, further comprising modeling an effect on a selected EIS signal in response to the concentration of the interferent determined to be within the body of the user, wherein modeling the effect on the selected EIS signal includes incorporating an imaginary component of the impedance signal, a counter voltage signal, and a current signal into a predictive model.

7. The method of claim 1, further comprising:
obtaining sensor glucose measurements with the sensing device; and
modeling an effect on sensor glucose measurements in response to the concentration of the interferent determined to be within the body of the user.

8. The method of claim 1, further comprising:
obtaining sensor glucose measurements with the sensing device;
modeling an effect on sensor glucose measurements in response to the concentration of the interferent determined to be within the body of the user; and
correcting the sensor glucose measurements based on the effect.

9. A method for correcting a sensor glucose measurement signal with a controller, the method comprising:
determining, with the controller, a concentration of an interferent in a body fluid;
modeling an effect on the sensor glucose measurement signal in response to the concentration of the interferent in the body fluid; and
correcting the sensor glucose measurement signal based on the modeled effect.

10. The method of claim 9 wherein determining, with the controller, a concentration of an interferent in a body fluid comprises receiving, with the controller, an input concentration of an interferent from a user.

11. The method of claim 9 wherein determining, with the controller, a concentration of an interferent in a body fluid comprises
storing a library of changes in electrochemical impedance spectroscopy (EIS) signals correlated to known concentrations of the interferent within bodies of study subjects, wherein the library is accessible to the controller;
monitoring EIS signals of a user with the controller; and
matching, with the controller, a change in an EIS signal of the user with a change in a selected EIS signal from the library.

12. The method of claim 11, wherein:
the library of changes in EIS signals is obtained at a selected frequency from about 0.1 to about 512 Hz; and
monitoring EIS signals of the user comprises monitoring EIS signals of the user at the same selected frequency.

13. The method of claim 11, wherein:
the library of changes in EIS signals correlated to known concentrations of the interferent within bodies of study subjects includes an imaginary component of the impedance signal;
monitoring EIS signals of the user with the controller comprises monitoring an imaginary component of the impedance signal of the user;
matching the change in an EIS signal of the user with a change in a selected EIS signal from the library comprises matching a change in the imaginary component of the impedance signal of the user with a change in a selected imaginary component of the impedance signal from the library.

14. The method of claim 11, wherein:
the library of changes in EIS signals correlated to known concentrations of the interferent within bodies of study subjects includes a counter voltage signal;
monitoring EIS signals of the user with the controller comprises monitoring a counter voltage signal of the user;
matching the change in a signal of the user with a change in a selected EIS signal from the library comprises matching a change in the counter voltage signal of the user with a change in a selected counter voltage signal from the library.

15. The method of claim 11, wherein modeling the effect on the sensor glucose measurement signal in response to the concentration of the interferent in the body fluid comprises incorporating imaginary components of impedance signals, counter voltage signals, and current signals into a predictive model.

16. An analyte monitoring apparatus comprising:
an electrochemical sensor for monitoring an electrochemical sensor placement site of a user, wherein the electrochemical sensor comprises a reference electrode; a counter electrode; and a working electrode;
a sensor input configured to receive signals from the electrochemical sensor; and
a processor coupled to the sensor input, wherein the processor is configured to characterize one or more signals received from electrodes of the electrochemical sensor and to determine a concentration of acetaminophen at the electrochemical sensor placement site.

17. The analyte monitoring apparatus of claim 16, wherein the processor is in communication with a library of electrochemical impedance spectroscopy (EIS) signals correlated to known concentrations of acetaminophen within bodies of study subjects, and wherein the processor is configured to monitor EIS signals of the user and to match a change in an EIS signal of the user with a change in a selected EIS signal from the library to determine the concentration of acetaminophen at the electrochemical sensor placement site.

18. The analyte monitoring apparatus of claim 16, wherein the processor is configured to model an effect on a sensor glucose measurement signal in response to the concentration of acetaminophen.

19. The analyte monitoring apparatus of claim 16, wherein the processor is configured to model an effect on a sensor glucose measurement signal in response to the concentration of acetaminophen and to correct the sensor glucose measurement signal based on the modeled effect.

20. The analyte monitoring apparatus of claim 16, wherein the processor is configured to model an effect on a sensor glucose measurement signal in response to the concentration of acetaminophen by incorporating an imaginary component of the impedance signal, a counter voltage signal, and a current signal into a predictive model.

21. A method for detecting the presence of an interferent in a body fluid, the method comprising:
contacting a sensing device with the body fluid;
transmitting a voltage to the sensing device;
monitoring a sensor signal from the sensing device in response to the voltage;
calculating a sensor signal rate of change; and
identifying when the sensor signal rate of change is greater than a threshold indicative of the presence of the interferent in the body fluid.

22. The method of claim 21, wherein:
monitoring the sensor signal from the sensing device in response to the voltage comprises taking a plurality of measurements of the sensor signal over a defined time period; and
calculating the sensor signal rate of change comprises determining the slope of the plurality of measurements of the sensor signal over a defined time period.

23. A method for operating a sensing device associated with a user, the sensing device including a controller coupled to a sensing element configured to measure a physiological condition in a body of the user, the method comprising:
transmitting a first voltage to the sensing device;
monitoring a first sensor signal from the sensing device in response to the first voltage;
learning, with the controller, that an interferent is in a body fluid;
in response to learning that the interferent is in the body fluid, transmitting a second voltage to the sensing device, wherein the second voltage is less than the first voltage; and
monitoring a second sensor signal from the sensing device in response to the second voltage.

24. The method of claim 23, wherein learning, with the controller, that the interferent is in the body fluid comprises receiving inputted information from the user.

25. The method of claim 23, wherein learning, with the controller, that the interferent is in the body fluid comprises:
calculating, with the controller, a first sensor signal rate of change; and
identifying when the first sensor signal rate of change is greater than a threshold indicative of the presence of the interferent in the body fluid.

26. The method of claim 25, wherein:
monitoring the first sensor signal from the sensing device in response to the first voltage comprises taking a plurality of measurements of the first sensor signal over a defined time period; and
calculating, with the controller, the first sensor signal rate of change comprises determining the slope of the plurality of measurements of the first sensor signal over a defined time period.

27. The method of claim 23 further comprising alternating application of the first voltage and the second voltage to the sensing device and monitoring the first sensor signal and the second sensor signal from the sensing device until detecting, with the controller, an absence of the interferent in the body fluid.

28. The method of claim 27 further comprising, after detecting the absence of the interferent in the body fluid, transmitting the first voltage to the sensing device and monitoring the first sensor signal from the sensing device in response to the first voltage.

29. The method of claim 27, wherein detecting the absence of the interferent in the body fluid comprises:
monitoring, with the controller, a first sensor signal rate of change; and
identifying when the first sensor signal rate of change is less than a threshold and an elapsed time period is met.

30. The method of claim 27, wherein alternating application of the first voltage and the second voltage to the sensing device comprises transmitting the first voltage to the sensing device for a first period of from about 1 to about 30 minutes and transmitting the second voltage to the sensing device for a second period of from about 1 to about 30 minutes.

31. The method of claim 23, wherein the first voltage is greater than 500 mV and the second voltage is less than 450 mV.
